(19) United States
(12) Patent Application Publication
(10) Pub. No.: US 2021/0161170 A1
(43) Pub. Date: Jun. 3, 2021

(54) A MIXTURE FOR THE PRODUCTION OF CHEWING GUMS

(71) Applicant: Milan Kadera, Liberec (CZ)

(72) Inventor: Milan Kadera, Liberec (CZ)

(21) Appl. No.: 17/046,340

(22) PCT Filed: Apr. 10, 2019
(86) PCT No.: PCT/CZ2019/000017

§ 371 Date: Oct. 9, 2020

(30) Foreign Application Priority Data
Apr. 13, 2018 (CZ) PU V2018-34869

(51) Int. Cl. A23G 4/06; A23G 4/10; A23G 4/08; A23L 29/00; A23L 29/25; A23L 29/30; A23L 33/105; A23L 33/12

(52) U.S. Cl. CPC A23G 4/068; A23G 4/10; A23G 4/08; A23G 4/066; A23L 33/12; A23L 29/25; A23L 29/37; A23L 33/105; A23L 29/04

(57) ABSTRACT

A mixture for the chewing gum which includes a powder-mixture and of a core composed of normal substances for the manufacture of chewing gum (sorbitol, Arabic gum, aromatic substances) and from the addition of the two active substances of caffeine and taurine in a concentration of caffeine from 0.77% to 3.75% and of taurine from 7.7% to 39.6% by weight. The taste of this mixture is deprived of a significant bitterness by the addition of Galium odoratum (woodruff) in a concentration of from 0.3% to 7% by weight, alternatively, it may be replaced by or supplemented with green tea from 0.2% to 4% by weight, where these additives reduce the bitterness of the mixture. It is also possible to add the above substances directly into the core, where, however, the concentration of the content of those substances is lower.

### CROSS REFERENCE TO RELATED APPLICATION

This application is a national stage entry of PCT/CZ2019/000017 filed Apr. 10, 2019, under the International Convention and claiming priority over Czech Republic Patent Application No. PU V2018-34869 filed Apr. 13, 2018.

### TECHNICAL FIELD

The technical solution relates to a chewing gum with powder filling and with a central molded core, filled with active ingredients for increased energy efficiency of the mixture.

### BACKGROUND ART

On the market there are currently some types of chewing gums containing stimulant substances such as caffeine, taurine or other substances with the stim effects. These known mixtures contain only a small concentration of such substances, e.g. 40-80 mg of caffeine without taurine or a maximum of 80 mg caffeine and 50 mg of taurine in a 4-5-gram piece of gum. Higher doses then cause the strong bitterness of the product and are basically inedible. None of so far known composition of a chewing gum mass does not contain such a concentration of caffeine and taurine, as they are contained these substances in 250 ml of energy drink.

From the known solutions we refer to the patent ZA201100433, which, however, primarily solves the powder filling enveloping the pressed core.

It is therefore clear that the chewing gums, as a stimulant supplement, currently are insufficiently effective and the consumer rather uses the energy drinks, where, however, there is a rapid onset of the effect, but also the rapid termination of this effect.

### DISCLOSURE OF THE INVENTION

The above deficiencies largely solves the composition of the mixture for the chewing gum according to this technical solution, which is composed of powder-mixture and of a core composed of normal substances for the manufacture of chewing gum (sorbitol, gum Arabic, aromatic substances) and from the addition of the two active substances of caffeine and taurine in a concentration of caffeine from 0.77% to 3.75% and of taurine from 7.7% to 39.6% by weight. The taste of this mixture is deprived of a significant bitterness by the addition of Galium odoratum (woodruff) in a concentration of from 0.3% to 7% by weight, alternatively, it may be replaced by or supplemented with green tea from 0.2% to 4% by weight, where these additives reduce the bitterness of the mixture. It is also possible to add the above substances directly into the core, where, however, the concentration of the content of those substances is lower.

The basic substances, namely caffeine and taurine, have in the mutual concentration the stimulant effect on the human organism.

### MODES FOR CARRYING OUT THE INVENTION

Exemplary composition of a chewing gum with a core and the powder mixture is obvious from the table:

Example 1

| Name of the ingredient | % |
|---|---|
| Maltitol P200 | 21.51 |
| Taurine | 21.29 |
| Maltitol P90 | 19.15 |
| Rubber base | 17.21 |
| Sorbitol | 9.98 |
| Maltitol syrup | 3.66 |
| Arabic gum | 2.30 |
| Caffeine | 1.97 |
| Aroma of sweet woodruff | 1.19 |
| Magnesium salts of fatty acids | 0.52 |
| Aroma of green tea | 0.36 |
| Dye: curcumin | 0.22 |
| Dye: paprika extract | 0.1 |
| Citric acid | 0.12 |
| Dye: carmine | 0.11 |
| Stevia | 0.08 |
| Sucralose | 0.07 |
| Aroma: Menthol crystal | 0.04 |
| Aroma: essential oil of peppermint | 0.04 |
| Carnauba wax | 0.04 |
| | 100.00 |

Example 2

| Name of the ingredient | % |
|---|---|
| Maltitol P200 | 11.20 |
| Taurine | 39.60 |
| Maltitol P90 | 11.15 |
| Rubber base | 17.21 |
| Sorbitol | 9.98 |
| Maltitol syrup | 3.66 |
| Arabic gum | 3.40 |
| Caffeine | 0.77 |
| Aroma of sweet woodruff | 1.19 |
| Magnesium salts of fatty acids | 0.52 |
| Aroma of green tea | 0.36 |
| Dye: curcumin | 0.22 |
| Dye: paprika extract | 0.13 |
| Citric acid | 0.12 |
| Dye: carmine | 0.11 |
| Stevia | 0.08 |
| Sucralose | 0.07 |
| Aroma: Menthol crystal | 0.04 |
| Aroma: essential oil of peppermint | 0.04 |
| Carnauba wax | 0.04 |
| | 100.00 |

Example 3

| Name of the ingredient | % |
|---|---|
| Maltitol P200 | 31.51 |
| Taurine | 7.70 |
| Maltitol P90 | 19.15 |
| Rubber base | 19.02 |
| Sorbitol | 9.98 |
| Maltitol syrup | 3.66 |
| Arabic gum | 2.30 |
| Caffeine | 3.75 |
| Aroma of sweet woodruff | 1.19 |
| Magnesium salts of fatty acids | 0.52 |
| Aroma of green tea | 0.36 |
| Dye: curcumin | 0.22 |
| Dye: paprika extract | 0.13 |
| Citric acid | 0.12 |
| Dye: carmine | 0.11 |
| Stevia | 0.08 |
| Sucralose | 0.07 |
| Aroma: Menthol crystal | 0.04 |
| Aroma: essential oil of peppermint | 0.04 |
| Carnauba wax | 0.04 |
| | 100.00 |

### INDUSTRIAL APPLICABILITY

This solution is usable in the manufacture of mixtures for the dietary supplements, primarily chewing gums.

1. A mixture for the production of chewing gums comprising:
0.77% to 3.75 by weight of caffeine; and
7.7% to 39.6% by weight of taurine:
wherein the mixture is added to a core made of a standard chewing gum.

2.The mixture for the production of chewing gums according to the claim 1, wherein the mixture further contains gallium odoratum in a concentration of from 0.3% to 7% by weight and/or green tea from 0.2% to 4% by weight.
